(19) United States
(12) Patent Application Publication
(10) Pub. No.: US 2002/0025917 A1
(43) Pub. Date: Feb. 28, 2002

(54) Compartmentalized device to enable a process of liquefying and administering aspirin as a first aid to heart attack victims

(76) Inventor: Edward Pappalardo, Albertson, NY (US)

Correspondence Address: EDWARD PAPPALARDO, 37 GARDEN DRIVE, ALBERTSON, NY, 11507, US

(21) Appl. No.: 09/839,277

(22) Filed: Apr. 23, 2001

Related U.S. Application Data
(60) Provisional application No. 60/228,305, filed on Aug. 28, 2000.

(51) Int. Cl. A61K 31/00; B65D 83/10

(52) U.S. Cl. 514/1; 206/363

(57) ABSTRACT

This invention relates to a novel process of creating an admixture of liquefied aspirin or other heart attack medication and administering said admixture to the buccal mucosa of the cheek pouch (mouth), and or the nasal passages of heart attack victims.

A process of delivering medication to the mucus and respiratory membranes of the mouth and or nasal passages and respiratory system, using a compartmentalized device whose contents will remain anhydrous until used.

This process is faster acting and more versatile than conventional oral or nasal medication, especially in emergencies where liquid admixtures would need to be prepared at the time of use.

The novelty of this invention is that it allows heart attack victim immediate access to a liquefied aspirin or heart attack medication. This is done with a convenient, pre packaged, compartmentalized device and applicator that is suitable for heart attack victims in a Varity of circumstances.

### CROSS REFERENCES TO RELATED APPLICATIONS

4778810 October 1998 Wenig

4885287 December 1989 Hussein

5122127 June 1992 Stanley

5458244 October 1995 Emori

5346061 September 1994 Newman

### BACKGROUND OF THE INVENTION

1. Field of Invention

The present invention relates to a fast and efficient method of administering liquid aspirin transorally/transnassaly as a first aid to heart attack victims. This medicament is mixed and administered by the use of a compartmentalized device containing a dose to effect ready to mix aspirin and liquid carrier

2. Background Art

It is accepted knowledge that the early administration of aspirin to a heart attack victim limits the damage to heart muscle due to the easing of platelet aggregation in the arteries. The suggested first aid for heart attack victums is the chewing of aspirin tablet. For various reasons, chewing a dry tablet can be a poor way of administering aspirin during a heart attack.

Many cases of heart attack will result in partial or total loss of consciousness making it difficult or impossible to administer a dry tablet.

In the remaining cases the physiological reaction to heart attacks will be stress and fear, causing a lack of saliva. Due to this lack of saliva, a substantial amount of aspirin tablet will remain unsoluable. The resulting large particles of tablet will slow or inhibit the absorption of the medication. If a heart attack victim chews a dry tablet with a lack of saliva they will be inclined to take liquid if available, this would encourage swallowing of the aspirin particles, and thus slow the rate of absorption.

This invention shows a concentrated and pharmaceutically effective dose of liquefied aspirin comprised of 325 mg. of crushed aspirin added to 1.25 ml. of a pharmaceutically acceptable liquid carrier. The components of the admixture are packaged in a compartmentalized device such as a small spray or dropper bottle, or an aerosol type sprayer.

The preferred administration of mecadent would be a spray application. This would allow the greatest area of contact to the buccal muccosa and or respiratory systems thus increasing the speed and efficiency. A dose to effect would take about 60 seconds.

The purpose of a compartmentalized device is due to the unstable nature of aspirin and similar medications that must remain anhydrous until used. These types of mecadents can not be liquefied and then stored for future use. The components of the compartmentalized device are of such weight and volume as when combined they are of a pharmaceutically effective dose.

Pre measured and ready to be mixed into a liquefied composition, this admixture allows a faster and more effective administration and absorption of the heart attack medication than a solid tablet. In addition, the concentration and volume of liquid medication 325 mg/1.25 ml is such that it allows the choice of oral and or nasal administration, minimizing loss from internal and external drainage.

In the preparation of first aid medication for heart attack victims, the level of consciousness of the victim can not be predicted. In the case of semi or unconscious heart attack victims, the administration of a nasal sprayed liquefied aspirin alone can be difficult or impossible due to drainage.

Furthermore, due to the irritating nature of a nasally administered concentrated dose of liquid aspirin the victim may involuntarily discharge significant amounts of medicament at this critical time.

Therefore, in these cases the oral administration of a pharmaceutically effective admixture of liquefied aspirin to the buccal mucosa of the cheek pouch will be the most effective system of delivery.

Prior art Hussain et al 4,885,287 shows the effective application of a liquefied aspirin delivered nasally for migraine and heart attack. However, in order to support his claim of sustained release, it is a slow delivery, in a controlled setting.

He shows no means of emergency preparation of admixture, nor does he realistically address the problem of semi or unconscious victims. Hussain does not expose the problem of loss due to drainage internally or externally due to the prior mentioned limitations of nasal administration. Hussain shows 300 mg of aspirin in four ml. of liquid, this is too voluminous for effective administration in a first aid heart attack situation.

Stanley 5,122,127 shows the effective oral absorption of medicaments by direct contact to the mucosal tissue with reference to:

Brown. “Absorption on Analgesics From the Buccal Muccous Membrane”, 196,The Practicioner, 125 (1966)

Dearden et al.,“A new Buccal Absorption Model”, and 23 J.Pharm.Pharmac. (1971)

Dollery et al.,“Differences in the Metabolism of Drugs Depending Upon There Routes of Administration”, 179,Annals of the New York Academy of Sciences, 108 (1971)

He shows no means of first aid application and like Hussain, reference is made to concentrations or volumes that would be inappropriate for emergency use.

Prior art recording compartmentalized containers are general in nature as related to this invention

Krause 6,036,005 shows a package for storing, mixing and dispensing multi component products.

No prior art shows a pre-packaged stable dosage of pharmaceutically effective dose of aspirin which is intended to be liquefied and administered to heart attack victims in the mouth and or nose

### SUMMARY

In view of the background, art it is apparent that the need exists for a process of delivering a concentrated pharmaceutically effective dose of liquefied aspirin transorally or transnassaly to heart attack victims.

It is a further object of this invention to provide and administer a pharmaceutically effective dose of liquid aspirin within 60 seconds to heart attack victims.

Accordingly, one object of the invention, due to the unstable nature of aspirin is to provide a ready to use admixture of liquid aspirin which will remain anhydrous until needed by packaging, said aspirin in a compartmentalized device.

Yet another object of this invention is to provide a method of administering liquid aspirin orally and or nasally in such concentrations and volume as to inhibit swallowing and increasing the rate of absorption through the buccal mucosa of the cheek pouch or nasal membranes.

Yet, another object of this invention is to show various compartmentalized devices and applicators for creating and delivering admixtures of liquid aspirins.

It is a further object of this invention to provide and administer a novel dosage form of liquefied aspirin to semi or unconscious heart attack victims specifically through the buccal mucosa of the cheek pouch by oral administration.

It is yet a further object of this invention to prepare and administer a dosage form of liquefied aspirin with the use of a compartmentalized device which would incorporate other beneficial components in aiding heart attack victims such as potassium, magnesium oxygen and other formulations that would be obvious to those skilled in the art.

Another object of the invention is to provide a coloring agent to signal a successful admixture of the blood thinning medication. This would also be used to signal a defect in packaging.

### BRIEF DESCRIPTION OF THE DRAWINGS

The figures are drawings of various types of compartmentalized devices and applicators for liquefying and dispensing single dosage forms of heart attack medication. The powder shown is 325 mg. of soluble aspirin and 1.25 ml. of common liquid carrier with or without pH adjusters.

### DETAILED DESCRIPTION OF THE INVENTION

The term“aspirin” as used refers to Salicylic Acid Acetate or Acetylsalicylic acid which has the structure ##STR1## aspirin formulations usually contains 300 to 500 mg per dosage unit.

The term “liquid carrier” refers to suitable pharmaceutically acceptable liquid carrier. The carrier is preferable purified water, with optional amounts of other minor ingredients such as pH adjusters

For those not skilled in the art of phamacutical formulations regarding liquid carriers reference is made to the text entitled “REMINGTONS PHARMACEUTICAL SCIENCES”, 17th edition, 1985, as well as many other recent publication.

In accord with the present invention, it has now been found that preparation of a liquid aspirin can remain stable and anhydrous if packaged in a compartmentalized device. Once liquefied in can be quickly administered to the buccal mucosa of the cheek pouch by oral administration or through the nasal/respiratory membranes. A single dose to effect of liquefied aspirin admixture is about 1.75 ml

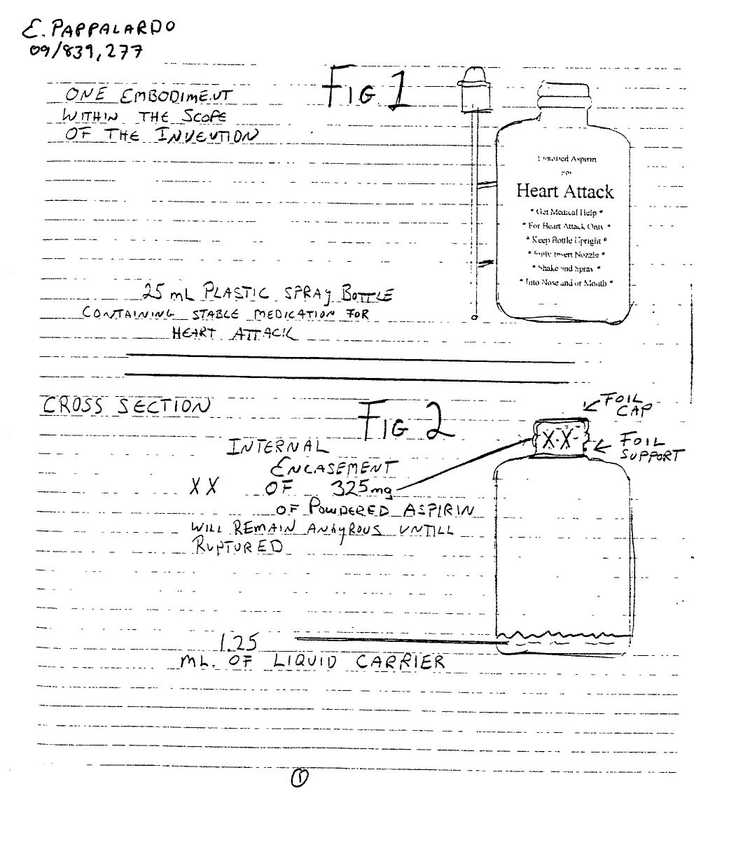

The invention shows a labeled small 25 ml spray bottle illustrated in (FIG. 1) containing a stable medication. It can be carried in a pocket, desk, glove compartment or any easy to reach convenient location.

There is no need to measure, crush, chew of obtain a source of liquid in order to take the medicament. Since the invention is a one-time dose to effect admixture, the long-term irritating properties of aspirin is not as much of a concern as was the loss of admixture due to drainage and swallowing. Therefore, liquid volume was minimized thus increasing the concentrations of aspirin and decreasing the time of absorption.

A preferred embodiment would be a small spray bottle, the atomization of admixture would cover a large area within the oral cavity or nasal passages giving the most efficient dose to effects rate of absorption.

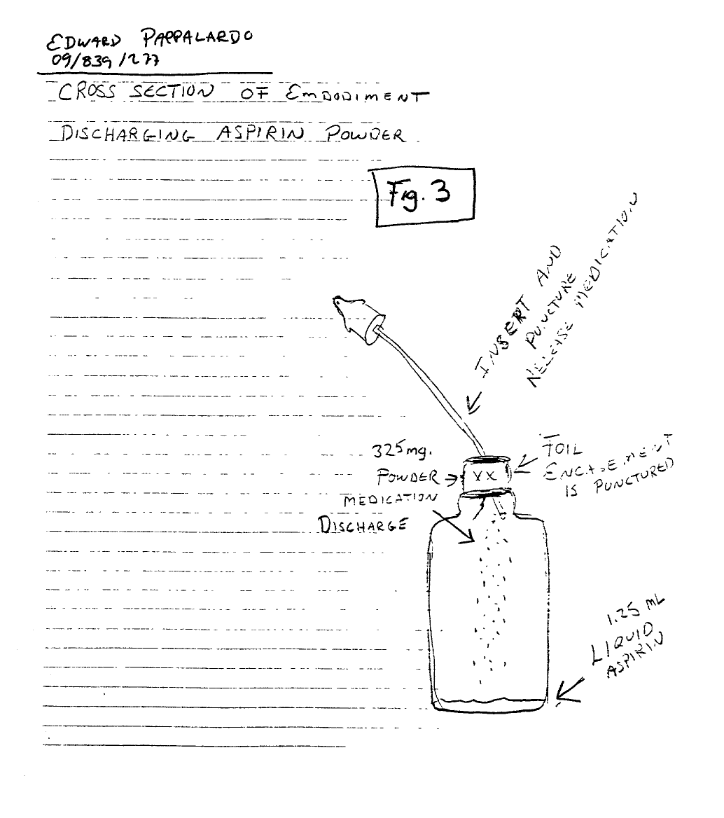

As seen in the cross sectional view of the embodiment illustrated in (FIG. 2) there is 325 mg of water soluble powdered aspirin encased in a waterproof compartment within the neck of the spray bottle. There is 1.25 ml of liquid carrier in the main body of spray bottle.

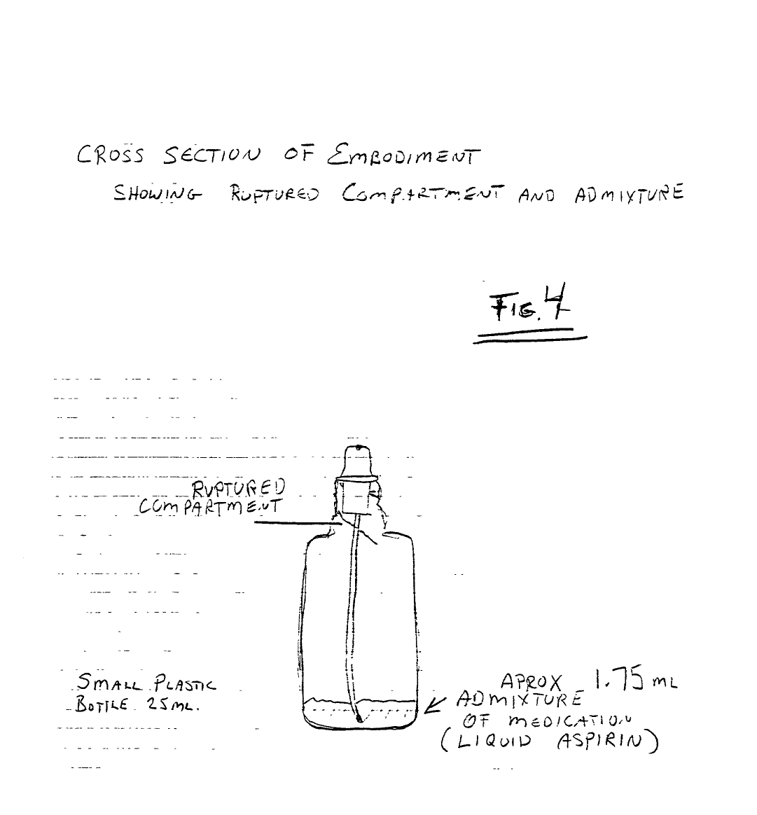

When the externally packaged nozzle/stem assemble in inserted through the top of the spray bottle (FIG. 3) the encasement will rupture and discharge the primary component (powdered aspirin) into the secondary component (liquid carrier).

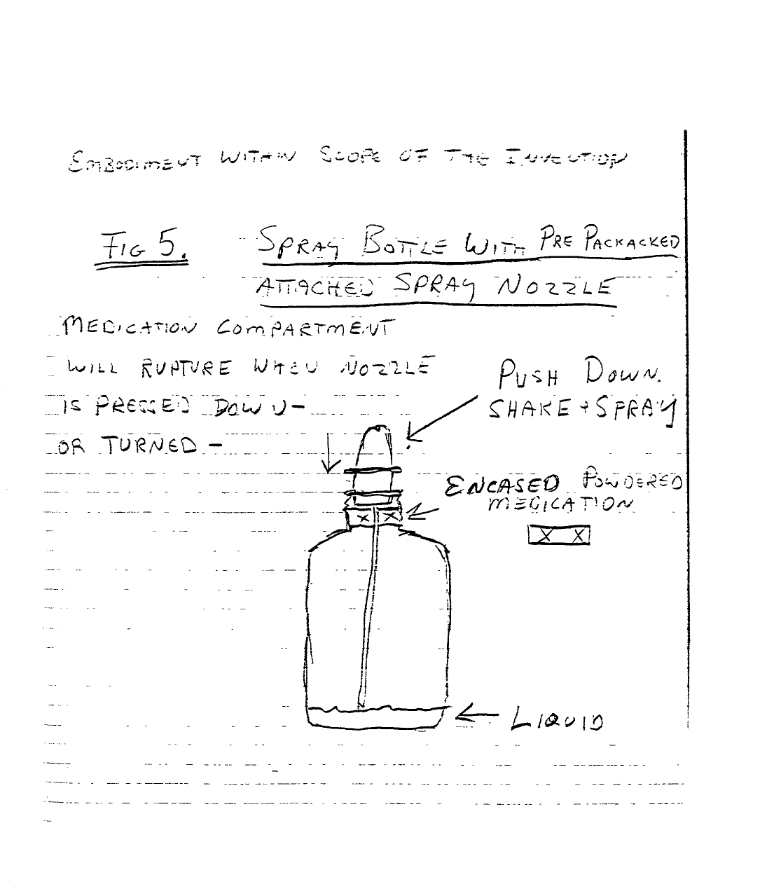

As the nozzle/stem assemble is completely pushed down it will lock and seal into the neck of the spray bottle (FIG. 4). The bottle is then manually shaken for 2-3 seconds creating a pharmaceutically effective ready to administer dose of heart attack medication.

A single dose to effect of liquefied aspirin would be administered by squeezing the spray bottle. In order to maximize absorpstion it would be administered in four applications approximately 10 second apart.

These four timed applications are critical for the most efficiently absorption and least loss of mecadent during administration.

Depending on the victim's condition the application of medicament could be done in various ways. The administration of admixture can be sprayed from this bottle into the oral cavity causing micro droplets of medicament to be dispersed onto the buccal muccosa of the cheek pouch where it would quickly be absorbed. Small remnants of medicament would also be absorbed sublingually with this method. The medicament could also be sprayed 4 times in 10 seconds intranasal or in combination with oral administration.

The liquid mecadent could also be administered orally with a spoon or other applicator. This could be done in the case of a defective nozzle assemble or in other such circumstances. In the case of a spoon applicator a ¼ tsp. Of liquid aspirin would be administered every 30 seconds until the complete dosage was finished this would take about 2 minutes for maximum absorption.

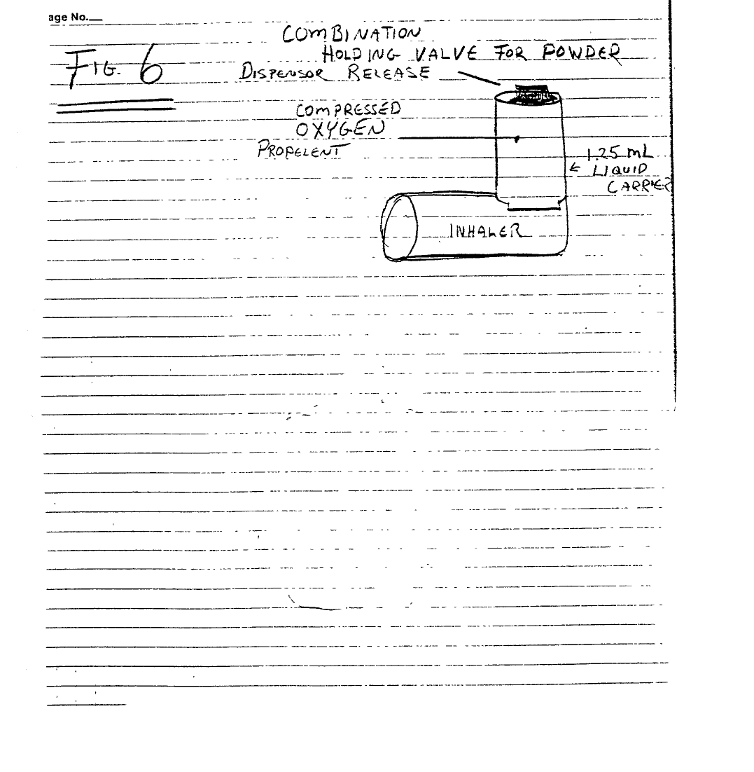

Various means of creating admixtures and rupturing compartments within a compartmentalized device are obvious. As seen in (FIG. 5) A spray bottle is packaged with a partially inserted nozzle/stem assembly. The primary component of powder is incased in the neck of the bottle with a seal around the inserted stem. When the nozzle is completely pushed down, it will rupture the compartment and release the primary component (aspirin) into the bottle as it simultaneously locks into the neck of the spray bottle.

There are also other obvious embodiments for those skilled in the art, such as when the base of the nozzle would perform as a holding valve for a powdered component and thus when turned would release said component. There are many other simple means of manipulating a compartmentalized device to assure an admixture of components.

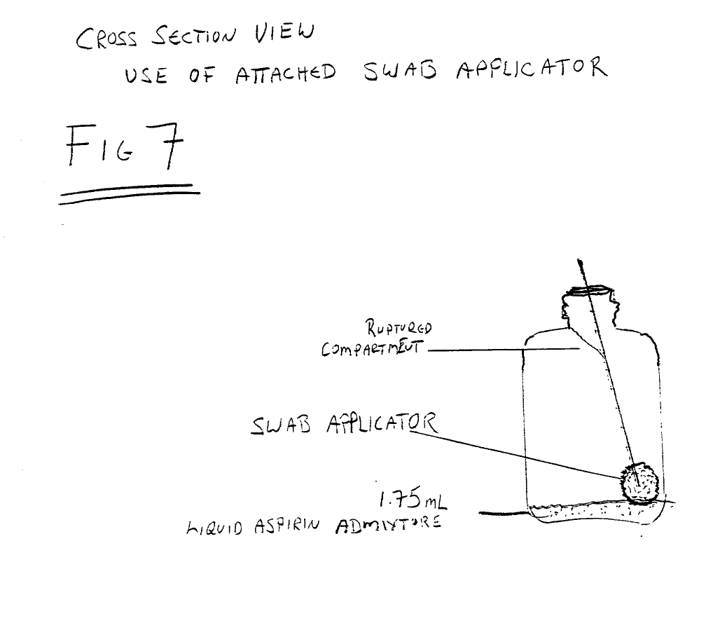

When an aerosol device is compartmentalized, it can also be used to create a phamacutacly effective dose of liquid aspirin. The cross sectional view of this embodiment within the scope of the invention (FIG. 6) shows a anhydrous encasement of powdered aspirin within a containment of liquid carrier before they are combined into a admixture.

The novelty of this embodiment is the optional addition of an oxygen propellant. With a simple regulator, the admixture of liquid aspirin will be dispensed in about 60 seconds, and the remainder of oxygen can be inhaled, further benefiting the heart attack victim.

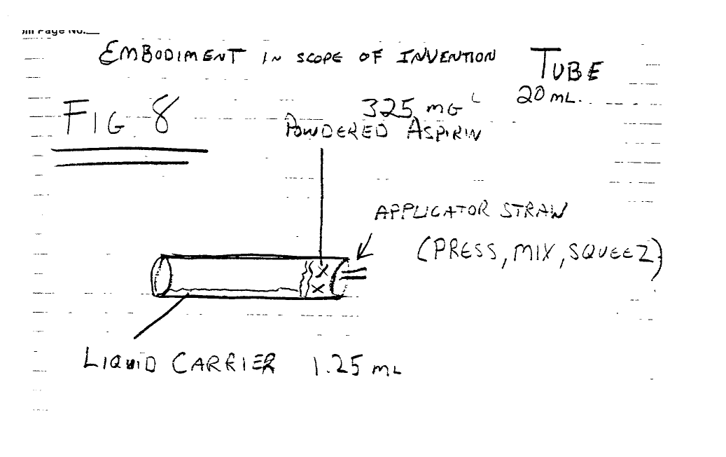

Various applicators can be used within the scope of the invention as seen in (FIG. 7) this shows a compartmentalized bottle with a swab applicator. The stem is used to rupture the primary compartment (powdered) causing it to discharge into the secondary component of liquid carrier. The bottle is shaken for 2-3 seconds to create an admixture and then the swab head inserted to absorb the mecadent. This wet swab is applied to the buccal mucossa of the heart attack victim repeatedly until the bottle is empty. This would take about 2 minutes. There are various compartmentalized containers that can be embodied within the scope of the invention such as a tube in (FIG. 8).

I claim:

1. A process of inhibiting platelet aggregation in heart attack victims by the administration of a pharmaceutically effective admixture of heart attack medication through the buccal mucosa of the cheek pouch (mouth) and or nasal membranes by the use of a compartmentalized device.

2. The pharmaceutically effective admixture of heart attack medication in claim 1, wherein said admixture is comprised of acetylsalicylic acid and a pharmaceutically acceptable liquid carrier.

3. The pharmaceutically effective dose of liquid aspirin in claim 2, wherein the aspirin remains anhydrous until ready to be mixed and administered.

4. The pharmaceutically effective admixture of heart attack medication in claim 1, wherein said admixture is that of a powder mixed with a liquid.

5. The pharmaceutically effective admixture of heart attack medication in claim 1, wherein said admixture would be that of a liquid mixed with a liquid.

6. The liquefied aspirin in claim 2, wherein the volume and concentration of said admixture is such that it would inhibit swallowing or dripping out rendering it less effective.

7. The pharmaceutically effective admixture of heart attack medication in claim 1, wherein said admixture is buffered to obtain an effective pH level in order to enhance absorption to the mucus and respiratory membranes.

8. The pharmaceutically effective admixture of heart attack medication in claim 1, wherein said admixture has a effervescent property to encourage a faster and more complete admixture.

9. The aspirin in claim 2, wherein the aspirin is in a water-soluble salt form for faster absorption when liquefied.

10. The medication in claim 1, wherein the medication is not limited to an aspirin based composition.

11. The pharmaceutically effective admixture of heart attack medication in claim 2 wherein beneficial components other then blood thing components are added.

12. The pharmaceutically effective admixture of heart attack medication in claim 2, wherein said liquid carrier is dimethyl isosorbide.

13. The compartmentalized device in claim 1, wherein the device dispenses a pharmaceutically effective dose of powdered blood-thinning medication.

14. The compartmentalized device in claim 1, wherein said device enables the admixture and dispensing of said heart attack medication by the use of various types of containers, applicator tips or attachments.

15. The compartmentalized device of claim 14, wherein the primary component of the admixture is encased in the top of the device and said casement is ruptured by the manipulation of the devices applicator allowing the contents of said encasement to internally discharge onto the secondary component of the device creating a pharmaceutically effective admixture.

16. The compartmentalized device of claim 14, wherein the manipulation of said applicator would be done by inserting an externally packaged applicator tip assembly into the top of the device containing the encased primary components of said admixture and causing said primary components to rupture and discharge into the secondary component of the inhaler.

17. The compartmentalized device of claim 14, wherein the manipulation of the devices applicator is done by pushing said applicator down and into the encased primary component of said admixture causing it to rupture and discharge into the secondary component of said admixture.

18. The compartmentalized device of claim 14, wherein the manipulation of said devices applicator is done by turning said applicator allowing the encased primary component to be released and discharge into the second component of said admixture.

19. The compartmentalized device of claim 14, wherein said device would offer a visual signal when the composition of medication was mixed and ready for immediate use or showing a defect in packaging by the same visual signal.
